(19) United States
(12) Patent Application Publication
(10) Pub. No.: US 2019/0175599 A1
(43) Pub. Date: Jun. 13, 2019

(54) TRPA1 ANTAGONIST FOR THE TREATMENT OF PAIN ASSOCIATED TO DIABETIC NEUROPATHIC PAIN

(71) Applicant: Glemark Pharmaceuticals S.A., La Chaux-de-Fonds (CH)

(72) Inventors: Neelima Khairatkar-Joshi, Thane (W) (IN); Monika Tandon, New Delhi (IN); Girish Gudi, Mumbai (IN); Patrick Keohane, London (GB); Zona Godsafe, Watford (GB)

(21) Appl. No.: 16/308,898

(22) PCT Filed: Sep. 16, 2015
(86) PCT No.: PCT/IB2015/057134

§ 371 Date: Dec. 11, 2018

(30) Foreign Application Priority Data
Sep. 16, 2014 (IN) 2956/MUM/2014
May 14, 2015 (IN) 1900/MUM/2015

(51) Int. Cl. A61K 31/519; A61K 9/00; A61P 29/02

(52) U.S. Cl. CPC A61K 31/519; A61P 29/02; A61K 9/0053

(57) ABSTRACT

The present patent application relates to a transient receptor potential ankyrin-1 (“TRPA1”) antagonist for the treatment of neuropathic pain in a subject. Particularly, the present patent application relates to a method of treating neuropathic pain in a subject in need thereof by orally administering to the subject a thienopyrimidinedione Compound as a TRPA1 antagonist. The present invention also relates to a pharmaceutical composition comprising the TRPA1 antagonist, and a process for preparing such a pharmaceutical composition.

### PRIORITY DOCUMENTS

This patent application claims priority to Indian Provisional Patent Applications number 2956/MUM/2014 filed on Sep. 16, 2014 and 1900/MUM/2015 filed on May 14, 2015, the contents of which are incorporated by reference herein.

### TECHNICAL FIELD OF THE INVENTION

The present patent application relates to a transient receptor potential ankyrin-1 (“TRPA1”) antagonist for the treatment of neuropathic pain in a subject. Particularly, the present patent application relates to a method of treating neuropathic pain in a subject in need thereof by orally administering to the subject a thienopyrimidinedione compound as a TRPA1 antagonist. The present invention also relates to a pharmaceutical composition comprising the TRPA1 antagonist, and a process for preparing such a pharmaceutical composition.

### BACKGROUND OF THE INVENTION

Pain is described as a complex constellation of unpleasant sensory, emotional and cognitive experiences provoked by real or perceived tissue damage and is manifested by certain autonomic, psychological and behavioral reactions and is a disease of epidemic proportions. From a neurobiological perspective, pain is believed to be of three different aspects: first, pain that is an early warning physiological protective system, essential to detect and minimize contact with damaging or noxious stimuli and is called ‘nociceptive pain’; second, pain that is adaptive and protective, by heightening sensory sensitivity after unavoidable tissue damage, which is mainly caused by activation of the immune system by tissue injury or infection and is normally called ‘inflammatory pain’; and the third type is pain which is not protective, but maladaptive resulting from abnormal functioning of the nervous system and generally called as ‘pathological pain’. This pathological pain is believed to be not a symptom of some disorder but rather a disease state of the nervous system, can occur after damage to the nervous system (neuropathic pain) or a situation where there is no such damage or inflammation (dysfunctional pain—like fibromyalgia, irritable bowel syndrome, temporomandibular joint disease, interstitial cystitis and other syndromes where there is substantial pain but no noxious stimulants and minimal/no peripheral inflammatory pathology).

Neuropathic pain is a pain caused by damage or disease that affects the somatosensory system. It may be associated with abnormal sensations called dysesthesia, and pain produced by normally non-painful stimuli (allodynia). Neuropathic pain may result from disorders of the peripheral nervous system or the central nervous system (brain and spinal cord). Thus, neuropathic pain may be divided into peripheral neuropathic pain, central neuropathic pain, or mixed (peripheral and central) neuropathic pain. Some treatment options for neuropathic pain include antidepressants (e.g. tricyclics and selective serotonin-norepinephrine reuptake inhibitors (SNRI's)), anticonvulsants (such as pregabalin, gabapentin, carbamazepine and oxcarbazepine and topical lidocaine. Opioid analgesics and tramadol are recognized as useful agents but are generally not recommended as first line treatments.

It is believed that TRPA1 is expressed in nociceptive neurons. Nociceptive neurons of the nervous system sense the peripheral damage and transmit pain signals. TRPA1 receptor is activated by a number of irritants that cause pain, including allyl isothiocyanate (AITC) and allicin, the pungent ingredients in mustard and garlic extracts, respectively; as well as α,β-unsaturated aldehydes, such as acrolein. The TRPA1 channel is the primary molecular site through which the pain pathway gets activated through an unusual mechanism involving covalent modification of cysteine and lysine residues within the N-terminal cytoplasmic domain of the channel protein. Thus, TRPA1 modulators are highly implicated in the alleviation of pain.

PCT Application Publication Nos. viz., WO 2004/055054, WO 2005/089206, WO 2007/073505, WO 2008/0949099, WO 2009/089082, WO2009/002933 WO 2009/158719, WO 2009/144548, WO 2010/004390, WO2010/109287, WO 2010/109334, WO 2010/109329, WO 2010/109328, WO2010/125469, WO 2010/004390, WO 2011/043954 and WO 2010/141805 describe various transient receptor potential (“TRP”) receptor modulators.

Many of the currently available pain therapies are either inadequate or cause uncomfortable to deleterious side effects and limitations. There are only three oral drugs (duloxetine, pregabalin and gabapentin) that have been formally approved in the Europe and the United States for treatment of painful DPN.

Adverse effects of currently available centrally acting DPN therapies and a low responder rate contribute to suboptimal clinical outcomes in a large proportion of subjects resulting in a high unmet need for specific treatments with better safety profile and novel approaches to identify subjects likely to respond to treatment. A need still exists for an effective treatment of neuropathic pain.

### SUMMARY OF THE INVENTION

The present invention relates to a method of treating neuropathic pain in a subject in need thereof by administering to the subject a therapeutically effective amount of a TRPA1 antagonist.

In one embodiment the present invention relates to a method of treating neuropathic pain in a subject in need thereof by orally administering to the subject a therapeutically effective amount of a TRPA1 antagonist of formula (A)

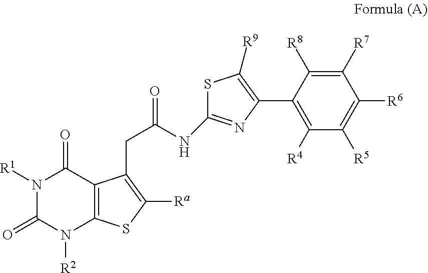

or a pharmaceutically acceptable salt thereof,
wherein

R¹ and R² are methyl;

R^{a} is hydrogen;

R⁴, R⁵, R⁶ and R⁷ are independently selected from hydrogen, fluoro, trifluoromethyl or trifluoromethoxy and

R⁸ and R⁹ is hydrogen.

Inter alia, the '930 application discloses a compound N-{4-[2,4-difluoro-3-(trifluoromethyl)phenyl]-1,3-thiazol-2-yl}-2-(1,3-dimethyl-2,4-dioxo-1,2,3,4-tetrahydrothieno[2,3-d]pyrimidin-5-yl)acetamide (hereinafter designated as “Compound I”) or its pharmaceutically acceptable salt.

In one embodiment, the present invention relates to a method of treating neuropathic pain in a subject in need thereof by orally administering to the subject a therapeutically effective amount of N-{4-[2,4-difluoro-3-(trifluoromethyl)phenyl]-1,3-thiazol-2-yl}-2-(1,3-dimethyl-2,4-dioxo-1,2,3,4-tetrahydrothieno[2,3-d]pyrimidin-5-yl)acetamide or its pharmaceutically acceptable salt.

In one embodiment, the present invention relates to a method of treating neuropathic pain in a subject in need thereof by orally administering to the subject a therapeutically effective amount of potassium salt of N-{4-[2,4-difluoro-3-(trifluoromethyl)phenyl]-1,3-thiazol-2-yl}-2-(1,3-dimethyl-2,4-dioxo-1,2,3,4-tetrahydrothieno[2,3-d]pyrimidin-5-yl)acetamide.

The therapeutically effective amount of Compound I or its pharmaceutically acceptable salt to be orally administered per day in context of the present invention is in the range from about 1 mg to about 1000 mg, or preferably from about 30 mg to about 800 mg, or more preferably from about 60 mg to about 600 mg. Typically, the therapeutically effective amount of Compound I or its pharmaceutically acceptable salt may be about 10 mg, or about 20 mg, or about 30 mg, or about 50 mg, or about 75 mg, or about 90 mg or about 100 mg, or about 120 mg, or about 150 mg, or about 180 mg, or about 200 mg, or about 250 mg, or about 300 mg, or about 350 mg, or about 400 mg, or about 450 mg, or about 500 mg. The therapeutically effective amount of Compound I or its pharmaceutically acceptable salt may be administered to the subject in the form of a pharmaceutical composition.

In the context of present invention, the effective amount of Compound I or its pharmaceutically acceptable salt may be orally administered as once daily, or in divided doses such as two/three/four times a day. Preferably Compound I or its pharmaceutically acceptable salt may be orally administered once daily or twice daily.

In one embodiment, the present invention relates a method of treating neuropathic pain in a subject in need thereof, the said method comprising orally administering per day to the subject from about 1 mg to about 1000 mg of Compound I or its pharmaceutically acceptable salt.

In one embodiment, the present invention relates a method of treating neuropathic pain in a subject in need thereof, the said method comprising orally administering per day to the subject from about 1 mg to about 1000 mg of Compound I or its pharmaceutically acceptable salt for a period of at least 4 weeks.

In one aspect of this embodiment the Compound I or its pharmaceutically acceptable salt is administered in an amount ranging from about 30 mg to about 800 mg, or from about 60 mg to about 600 mg daily. In another aspect of this embodiment, the Compound I or its pharmaceutically acceptable salt is administered once daily or twice daily.

In yet another aspect of this embodiment, the neuropathic pain comprises pain associated with peripheral neuropathy, diabetic peripheral neuropathy (DPN), trigeminal neuralgia, post herpetic neuralgia, visceral pain, cancer, multiple sclerosis, spinal-cord injury, fibromyalgia, stroke, inflammatory disorder, mechanical hyperalgesia, cold allodynia. Preferably the neuropathic pain comprises pain associated with peripheral neuropathy, diabetic peripheral neuropathy, post herpetic neuralgia, spinal-cord injury, fibromyalgia, mechanical hyperalgesia, and cold allodynia. Most preferably, the neuropathic pain comprises pain associated with diabetic peripheral neuropathy.

In an embodiment, the Compound I is administered in the form of its potassium salt.

In one embodiment the present invention relates to a method of treating pain associated with diabetic peripheral neuropathy by orally administering to the subject a therapeutically effective amount of N-{4-[2,4-difluoro-3-(trifluoromethyl)phenyl]-1,3-thiazol-2-yl}-2-(1,3-dimethyl-2,4-dioxo-1,2,3,4-tetrahydrothieno[2,3-d]pyrimidin-5-yl)acetamide or its pharmaceutically acceptable salt.

In one embodiment the present invention relates to a method of treating pain associated with diabetic peripheral neuropathy in a subject in need thereof by orally administering to the subject a therapeutically effective amount of potassium salt of N-{4-[2,4-difluoro-3-(trifluoromethyl)phenyl]-1,3-thiazol-2-yl}-2-(1,3-dimethyl-2,4-dioxo-1,2,3,4-tetrahydrothieno[2,3-d]pyrimidin-5-yl)acetamide.

In one embodiment the present invention relates to a method of treating pain associated with diabetic peripheral neuropathy in a subject in need thereof, said method comprising orally administering to the subject from about 30 mg to about 800 mg, or preferably from about 60 mg to about 600 mg of Compound I or its pharmaceutically acceptable salt.

In one embodiment the present invention relates to a method of treating pain associated with diabetic peripheral neuropathy wherein the Compound I or its pharmaceutically acceptable salt is administered once daily or twice daily.

In one embodiment the present invention relates to a method of treating pain associated with diabetic peripheral neuropathy in a subject in need thereof, said method comprising orally administering to the subject from about 30 mg to about 800 mg, or preferably from about 60 mg to about 600 mg of Compound I or its pharmaceutically acceptable salt per day for a period of at least 4 weeks.

In an aspect of this embodiment, the Compound I or its pharmaceutically acceptable salt is administered once daily or twice daily. In another aspect, the Compound I is administered in the form of its potassium salt.

In an embodiment, the present invention relates to a method of treating pain associated with diabetic peripheral neuropathy in a subject in need thereof, said method comprising orally administering to the subject about 250 mg of Compound I or its pharmaceutically acceptable salt, preferably twice daily.

In an embodiment, the present invention relates to a method of treating pain associated with diabetic peripheral neuropathy in a subject in need thereof, said method comprising orally administering to the subject about 250 mg of Compound I or its pharmaceutically acceptable salt twice daily for a period of at least 4 weeks.

In one embodiment the present invention relates to a method of treating pain associated with diabetic peripheral neuropathy in a subject in need thereof, said method comprising orally administering to the subject Compound I or its pharmaceutically acceptable salt wherein the subject showed more than 30% reduction or preferably more than 50% reduction from baseline in average pain intensity (API) score.

In one embodiment the present invention relates to a method of treating pain associated with diabetic peripheral neuropathy in a subject in need thereof, said method comprising orally administering to the subject Compound I or its pharmaceutically acceptable salt wherein the subject has preserved peripheral nerve function.

In this embodiment Compound I or its pharmaceutically acceptable salt is administered to a subject about 30 mg to about 800 mg, or from about 60 mg to about 600 mg.

In this embodiment Compound I or its pharmaceutically acceptable salt is administered once daily or twice daily.

In this embodiment Compound I or its pharmaceutically acceptable salt is administered to a subject about 30 mg or about 90 mg or about 250 mg.

In an embodiment, the present invention relates to a method of treating pain associated with diabetic peripheral neuropathy in a subject in need thereof, said method comprising orally administering to the subject Compound I or its pharmaceutically acceptable salt, wherein said method is further characterized by one or more of the followings:
A change in average pain intensity (API) ranging from about 10% to about 50%, or from about 25% to about 35% from baseline.
A change in night-time average pain intensity ranging from about 10% to about 50%, or from about 25% to about 35% from baseline.
A change in night-time worst pain intensity ranging from about 10% to about 50%, or from about 25% to about 35% from baseline.
A change in mean sleep interference score ranging from about 20% to about 60%, or from about 35% to about 45% from baseline.
A reduction in mean daily dose of rescue medication ranging from about 50% to about 90%, or from about 60% to about 80% from baseline.
An onset of sustained improvement (Reduction by ≥2 points) in the 24-hour daily average pain intensity score is achieved in about 20 days or in about 18 days from the baseline.

In an aspect of this embodiment, the subject is administered about 250 mg of Compound I or its pharmaceutically acceptable salt.

In another aspect of this embodiment, the subject is administered Compound I or its pharmaceutically acceptable salt twice daily.

In an aspect of this embodiment the subject is orally administered per day from about 30 mg to about 800 mg or from about 60 mg to about 600 mg of Compound I or its pharmaceutically acceptable salt.

In an aspect of this embodiment is administered about 250 mg of Compound I or its pharmaceutically acceptable salt.

In an aspect of this embodiment subject is administered Compound I or its pharmaceutically acceptable salt once daily or twice daily

In an aspect of this embodiment subject is administered about 250 mg of Compound I or its pharmaceutically acceptable salt once daily or twice daily.

In one aspect of this embodiment the subjects showed more than 30% reduction or preferably more than 50% reduction from baseline in average pain intensity (API) score.

In one aspect of this embodiment the subject to be treated for pain associated with diabetic peripheral neuropathy is characterized by one or more of the followings:
A subject having moderate to severe pain with a baseline 24-hour average daily pain intensity score of 5-8 as measured on 11-point pain intensity numeric rating scale (NRS).
A subject having preserved peripheral sensory nerves (i.e., non-denervation subgroup excluding subjects with relatively damaged peripheral sensory neurons detected by Qualitative Sensory Test (QST)).
A subject having hypersensitivity to cold stimulus and/or mechanical stimulus.

In another embodiment of the present invention, the subject to be treated for diabetic peripheral neuropathy is characterized by one or more of the followings:
A human male or female subject aged between 18-75 years.
A subject having diabetes mellitus (type 1 or 2) with a distal symmetric chronic senorimetor painful peripheral neuropathy.
A subject having history of diabetic peripheral neuropathy for at least 6 months.
A subject having “Douleur Neuropathique en 4” (DN4) questions score of ≥4.
A subject having a baseline 24-hour average daily pain intensity score ≥4 and <9 as measured on a 11-point pain intensity numeric rating scale (NRS).
A subject having stable glycemic control (i.e., Glycosylated Hemoglobin (HbAlc) <8%) for at least one or two or three months.
A subject detected to have mechanical hyperalgesia and/or cold allodynia.

In one embodiment the present invention relates to a method of treating pain associated with diabetic peripheral neuropathy in a subject in need thereof, said method comprising orally administering to the subject about 250 mg of Compound I or its pharmaceutically acceptable salt twice daily for a period of at least 4 weeks, wherein more than 30% reduction or preferably more than 50% reduction from baseline in average pain intensity (API) score is observed in the subject.

In another aspect of this embodiment the subject is characterized by one or more of the followings:
A subject having moderate to severe pain with a baseline 24-hour average daily pain intensity score of 5-8 as measured on 11-point pain intensity numeric rating scale (NRS).
A subject having preserved peripheral sensory nerves (i.e., non-denervation subgroup excluding subjects with relatively damaged peripheral sensory neurons detected by Qualitative Sensory Test (QST)).
A subject having hypersensitivity to cold stimulus and/or mechanical stimulus.

In one embodiment the present invention relates to a method of treating pain associated with diabetic peripheral neuropathy in a subject in need thereof comprising orally administering to the subject a therapeutically effective amount of Compound I or its pharmaceutically acceptable salt, wherein the mean steady state maximum plasma concentration (Cₘₐₓ) of the Compound I or its pharmaceutically acceptable salt in the subject is in the range of about 300 ng/ml to about 5500 ng/ml at about 0.0 hour to about 24 hours after repeated oral administration at steady state. Preferably, the mean steady state maximum plasma concentration (Cₘₐₓ) of the Compound I or its pharmaceutically acceptable salt in the subject ranges from about 1000 ng/ml to about 5300 ng/ml, or about 1800 ng/ml to about 3800 ng/ml at about 0 hour to about 24 hours after repeat oral administration at steady state.

In another embodiment the present invention relates to a method of treating pain associated with diabetic peripheral neuropathy in a subject in need thereof comprising orally administering to the subject a therapeutically effective amount of Compound I or its pharmaceutically acceptable salt, wherein the mean steady state exposure (AUC 0_24) of the Compound I or its pharmaceutically acceptable salt in the subject ranges from about 3,000 ng.hr/ml to about 55,000 ng.hr/ml. Preferably, the mean steady state exposure (AUC 0_24) of the Compound 1 or its pharmaceutically acceptable salt in the subject ranges from about 15,000 ng.hr/ml to about 45,000 ng.hr/ml, or about 25,000 ng.hr/ml to about 40,000 ng.hr/ml.

In one embodiment the present invention relates to a method of treating pain associated with diabetic peripheral neuropathy in a subject in need thereof, the said method comprising orally administering to the subject a pharmaceutical composition comprising from about 30 mg to about 800 mg, or preferably from about 60 mg to about 600 mg of Compound I or its pharmaceutically acceptable salt for a period of at least 4 weeks. In one aspect, the pharmaceutical composition comprises 250 mg of Compound I or its pharmaceutically acceptable salt.

In an aspect of this embodiment, the pharmaceutical composition is administered once daily or twice daily. In another aspect, the pharmaceutical composition comprises Compound I as potassium salt.

In an aspect of this embodiment, the present invention relates to a method of treating pain associated with diabetic peripheral neuropathy in a subject having preserved peripheral nerve function, said method comprising orally administering to the subject a Compound I or its pharmaceutically acceptable salt.

In an embodiment, the present invention relates to a method of treating pain associated with diabetic peripheral neuropathy in a subject having preserved peripheral nerve function, said method comprising orally administering to the subject about 30 mg to about 800 mg, or from about 60 mg to about 600 mg of Compound I or its pharmaceutically acceptable salt.

In an embodiment, the present invention relates to a method of treating pain associated with diabetic peripheral neuropathy in a subject having preserved peripheral nerve function, said method comprising orally administering to the subject about 250 mg of Compound I or its pharmaceutically acceptable salt.

In an embodiment, the present invention relates to a method of treating pain associated with diabetic peripheral neuropathy in a subject having preserved peripheral nerve function, said method comprising orally administering to the subject about 30 mg to about 800 mg, or from about 60 mg to about 600 mg of Compound I or its pharmaceutically acceptable salt once daily or twice daily.

In an embodiment, the present invention relates to a method of treating pain associated with diabetic peripheral neuropathy in a subject having preserved peripheral nerve function, said method comprising orally administering to the subject about 250 mg of Compound I or its pharmaceutically acceptable salt once daily or twice daily.

In an embodiment, the present invention relates to a method of treating pain associated with diabetic peripheral neuropathy in a subject having preserved peripheral nerve function, said method comprising orally administering to the subject about 250 mg of Compound I or its pharmaceutically acceptable salt once daily or twice daily for a period of at least 4 weeks.

In an embodiment, the present invention relates to a method of treating pain associated with diabetic peripheral neuropathy in a subject having preserved peripheral nerve function, said method comprising orally administering to the subject Compound I or its pharmaceutically acceptable salt, wherein change in average pain intensity (API) ranging from about 20% to about 70%, or from about 35% to about 45% from baseline.

In one aspect of this embodiment, a subject is further characterized by one or more of the following:
A subject having baseline average pain intensity (API) score of ≥5
Cold detection threshold >18° C. at baseline as determined by QST.
Warm detection threshold <49° C. at baseline as determined by QST.

In another aspect of this embodiment the subjects showed more than 30% reduction or preferably more than 50% reduction from baseline in average pain intensity (API) score.

In one aspect of this embodiment the Compound I or its pharmaceutically acceptable salt is administered in an amount ranging from about 30 mg to about 800 mg, or from about 60 mg to about 600 mg daily. In another aspect of this embodiment, the Compound I or its pharmaceutically acceptable salt is administered once daily or twice daily.

In one aspect of this embodiment the Compound I or its pharmaceutically acceptable salt is administered about 250 mg. In another aspect of this embodiment, the Compound I or its pharmaceutically acceptable salt is administered once daily or twice daily.

In one aspect of this embodiment the Compound I or its pharmaceutically acceptable salt is administered about 250 mg. In another aspect of this embodiment, the Compound I or its pharmaceutically acceptable salt is administered once daily or twice daily for a period of at least 4 weeks.

In an embodiment, the present invention relates to a method of treating pain associated with diabetic peripheral neuropathy in a subject having preserved peripheral nerve function, said method comprising orally administering to the subject Compound I or its pharmaceutically acceptable salt once daily or twice daily for a period of at least 4 weeks or 12 weeks.

In one embodiment the present invention relates to a method of treating pain associated with diabetic peripheral neuropathy in a subject having preserved peripheral nerve function, the said method comprising orally administering to the subject a pharmaceutical composition comprising from about 30 mg to about 1000 mg, or from about 150 mg to about 800 mg, or from about 300 mg to about 600 mg of Compound I or its pharmaceutically acceptable salt.

In one embodiment the present invention relates to a method of treating pain associated with diabetic peripheral neuropathy in a subject having preserved peripheral nerve function, the said method comprising orally administering to the subject a pharmaceutical composition comprising from about 20 mg to about 1000 mg, or from about 30 mg to about 800 mg, or from about 60 mg to about 600 mg of Compound I or its pharmaceutically acceptable salt.

In one embodiment the present invention relates to a method of treating pain associated with diabetic peripheral neuropathy in a subject having preserved peripheral nerve function, the said method comprising orally administering to the subject a pharmaceutical composition comprising from about 20 mg to about 1000 mg, or from about 30 mg to about 800 mg, or from about 60 mg to about 600 mg of Compound I or its pharmaceutically acceptable salt once daily or twice daily.

In one embodiment the present invention relates to a method of treating pain associated with diabetic peripheral neuropathy in a subject having preserved peripheral nerve function, the said method comprising orally administering to the subject a pharmaceutical composition comprising from about 20 mg to about 1000 mg, or from about 30 mg to about 800 mg, or from about 60 mg to about 600 mg of Compound I or its pharmaceutically acceptable salt for a period of at least 12 weeks.

In an aspect of this embodiment, the pharmaceutical composition is administered once daily or twice daily. In another aspect, the pharmaceutical composition comprises Compound I as potassium salt.

In an embodiment, the present invention relates to a method of treating pain associated with diabetic peripheral neuropathy in a subject having preserved peripheral nerve function, said method comprising orally administering to the subject Compound I or its pharmaceutically acceptable salt in the form of granules or tablet formulation at three different dose levels selected from 30 mg, 90 mg, 250 mg, 500 mg and 750 mg or 1000 mg.

In an embodiment, the present invention relates to a method of treating pain associated with diabetic peripheral neuropathy in a subject having preserved peripheral nerve function, said method comprising orally administering to the subject Compound I or its pharmaceutically acceptable salt in the form of granules or tablet formulation at three different dose levels selected from 30 mg, 90 mg, 250 mg, 500 mg and 750 mg or 1000 mg once daily or twice daily.

In an embodiment, the present invention relates to a method of treating pain associated with diabetic peripheral neuropathy in a subject having preserved peripheral nerve function, said method comprising orally administering to the subject Compound I or its pharmaceutically acceptable salt in the form of granules or tablet formulation at three different dose levels selected from 30 mg, 90 mg, 250 mg, 500 mg and 750 mg or 1000 mg once daily or twice daily for a period of at least 12 weeks.

In an embodiment, the present invention relates to a method of treating pain associated with diabetic peripheral neuropathy in a subject having preserved peripheral nerve function, said method comprising orally administering to the subject Compound I or its pharmaceutically acceptable salt in the form of granules or tablet formulation at three different dose levels selected from 30 mg, 90 mg, 250 mg, 500 mg and 750 mg or 1000 mg once daily or twice daily for a period of at least 12 weeks, wherein said method comprises determination of one or more of the followings:
A change in average pain intensity (API) from baseline to day 85 based on 11-point pain intensity NRS.
A change from baseline to day 15, 29, 57, 85 and 112 in
   Night-time average pain intensity
   Night-time worst pain intensity
   Mean sleep interference score
   A reduction in mean daily dose of rescue medication

In one aspect of this embodiment subjects having preserved peripheral nerve function showed more than 30% reduction or preferably more than 50% reduction from baseline in average pain intensity (API) score.

In another embodiment of the present invention, the subject having preserved peripheral nerve function to be treated for pain associated with diabetic peripheral neuropathy is characterized by one or more of the followings:
A human male or female subject aged between 18-75 years having diabetes mellitus (type 1 or 2) with a distal symmetric chronic sensorimotor painful peripheral neuropathy.
A subject having history of diabetic peripheral neuropathy for at least 6 months and no greater than 5 years.
A subject having the normal reference range 5 for thermal detection thresholds (CDT and WDT) based on QST.
A subject having “Douleur Neuropathique en 4” (DN4) questions score of ≥4.
A subject having a baseline 24-hour average daily pain intensity score ≥5 and <9 as measured on 11-point pain intensity numeric rating scale (NRS).
A subject being treatment naïve or one with pain not adequately controlled with up to maximum of 2 medications for treatment of pain.
A subject having Glycosylated Hemoglobin (HbAlc) level up to 11% and is stable on anti-diabetic medication/s for at least 3 months prior to screening.

In one embodiment, the present invention relates to a method of treating pain associated with diabetic peripheral neuropathy in a subject having preserved peripheral nerve function, comprising orally administering to the subject in need thereof a therapeutically effective amount of Compound I or its pharmaceutically acceptable salt, wherein the mean steady state maximum plasma concentration (Cₘₐₓ) of the Compound I or its pharmaceutically acceptable salt in the subject is in the range of about 300 ng/ml to about 5500 ng/ml at about 0.0 hour to about 24 hours after repeat oral administration at steady state. Preferably, the mean steady state maximum plasma concentration (Cₘₐₓ) of the Compound I or its pharmaceutically acceptable salt in the subject ranges from about 1000 ng/ml to about 5300 ng/ml, or about 1800 ng/ml to about 3800 ng/ml at about 0 hour to about 24 hours after repeat oral administration, at steady state.

In another embodiment, the present invention relates to a method of treating pain associated with diabetic peripheral neuropathy in a subject having preserved peripheral nerve function, said method comprising orally administering to the subject in need thereof a therapeutically effective amount of Compound I or its pharmaceutically acceptable salt, wherein the mean steady state exposure (AUC ₀_{_}₂₄) of the Compound I or its pharmaceutically acceptable salt in the subject ranges from about 3,000 ng.hr/ml to about 55,000 ng.hr/ml. Preferably, the mean steady state exposure (AUC ₀_{_}₂₄) of the Compound I or its pharmaceutically acceptable salt in the subject ranges from about 15,000 ng.hr/ml to about 45,000 ng.hr/ml, or about 25,000 ng.hr/ml to about 40,000 ng.hr/ml.

In one embodiment, the present invention relates to a method of treating diabetic peripheral neuropathy in a subject in need thereof, said method comprising orally administering to the subject in need thereof a therapeutically effective amount of Compound I or its pharmaceutically acceptable salt wherein the method results in a disease modifying effect as assessed by a sustained reduction in pain for a period of at least 6 weeks after termination of the treatment.

In one embodiment, the present invention relates to a method of treating pain associated with diabetic peripheral neuropathy in a subject in need thereof, said method comprising orally administering to the subject a therapeutically effective amount of Compound I or its pharmaceutically acceptable salt, wherein a subject is on stable maintenance dose of at least one another diabetic peripheral neuropathic pain medication and have a moderate to severe pain. In one aspect, the stable maintenance dose levels have been stable for at least 30 days prior to the screening visit. In one aspect, another diabetic peripheral neuropathic pain medication may include pregabalin, gabapentin, duloxetine, Capsaicin or salts thereof.

In one embodiment, the present invention relates to a method of treating pain associated with diabetic peripheral neuropathy in a subject in need thereof, said method comprising orally administering to the subject a therapeutically effective amount of Compound I or its pharmaceutically acceptable salt, wherein a subject is nonresponsive to other diabetic peripheral neuropathic pain medication In one aspect a subject is having preserved peripheral nerve function.

In one embodiment, the present invention relates to a method of treating pain associated with diabetic peripheral neuropathy in a subject in need thereof, said method comprising orally administering to the subject a therapeutically effective amount of Compound I or its pharmaceutically acceptable salt, wherein a subject is nonresponsive to other diabetic peripheral neuropathic pain medication such as pregabalin, gabapentin, duloxetine, Capsaicin or salts thereof. In one aspect a subject is having preserved peripheral nerve function.

In one embodiment, the present invention relates to a method of treating pain associated with diabetic peripheral neuropathy in a subject in need thereof, said method comprising orally administering to the subject a therapeutically effective amount of Compound I or its pharmaceutically acceptable salt, wherein a subject is inadequately responsive to other diabetic peripheral neuropathic pain medication such as pregabalin, gabapentin, duloxetine, Capsaicin or salts thereof. In one aspect a subject is having preserved peripheral nerve function.

In one embodiment, the present invention relates to a method of treating pain associated with diabetic peripheral neuropathy by orally administering to the subject a therapeutically effective amount of N-{4-[2,4-difluoro-3-(trifluoromethyl)phenyl]-1,3-thiazol-2-yl}-2-(1,3-dimethyl-2,4-dioxo-1,2,3,4-tetrahydrothieno[2,3-d]pyrimidin-5-yl)acetamide (“Compound I”) or its pharmaceutically acceptable salt wherein the subject is on pre medication of another diabetic peripheral neuropathic pain medication. In this embodiment, another diabetic peripheral neuropathic pain medication includes but not limited to pregabalin, gabapentin, duloxetine, Capsaicin or salts thereof.

In one embodiment the present invention relates to a method of treating pain associated with diabetic peripheral neuropathy in a subject in need thereof, said method comprising orally administering to the subject Compound I or its pharmaceutically acceptable salt, wherein no treatment related adverse effects have been observed. In one aspect a subject is having preserved peripheral nerve function.

In one embodiment the present invention relates to a method of treating pain associated with diabetic peripheral neuropathy in a subject in need thereof, said method comprising orally administering to the subject about 250 mg of Compound I or its pharmaceutically acceptable salt once daily or twice daily, wherein no treatment related adverse effects have been observed. In one aspect a subject is having preserved peripheral nerve function.

In one embodiment the present invention relates to a method of treating pain associated with diabetic peripheral neuropathy in a subject in need thereof, said method comprising orally administering to the subject about 250 mg of Compound I or its pharmaceutically acceptable salt once daily or twice daily for a period of at least 4 weeks, wherein no treatment related adverse effects have been observed. In one aspect a subject is having preserved peripheral nerve function.

In one embodiment the present invention relates to a method of alleviating symptoms of painful diabetic peripheral neuropathy in a subject in need thereof, said method comprising orally administering to the subject therapeutically effective amount of Compound I or its pharmaceutically acceptable salt. In one aspect a subject is having preserved peripheral nerve function.

In one embodiment the present invention relates to a method of slowing the appearance of symptoms of painful diabetic peripheral neuropathy in a subject in need thereof, said method comprising orally administering to the subject therapeutically effective amount of Compound I or its pharmaceutically acceptable salt. In one aspect a subject is having preserved peripheral nerve function.

In one embodiment the present invention relates to a method of slowing the progression of symptoms of painful diabetic peripheral neuropathy in a subject in need thereof, said method comprising orally administering to the subject therapeutically effective amount of Compound I or its pharmaceutically acceptable salt. In one aspect a subject is having preserved peripheral nerve function.

In an embodiment the present invention relates to a method of relieving pain associated with diabetic peripheral neuropathy by administering a therapeutically effective amount of Compound I or its pharmaceutically acceptable salt in preparation of a pharmaceutical composition suitable for oral administration in a subject in need thereof. In one aspect a subject is having preserved peripheral nerve function.

In an embodiment, the present invention also relates to Compound I or its pharmaceutically acceptable salt in a therapeutically effective amount for the treatment of pain associated with diabetic peripheral neuropathy in a subject in need thereof. In one aspect a subject is having preserved peripheral nerve function.

In an embodiment, the present invention relates to use of a therapeutically effective amount of Compound I or its pharmaceutically acceptable salt in preparation of a pharmaceutical composition suitable for oral administration for the treatment for pain associated with diabetic peripheral neuropathy in a subject in need thereof. In one aspect a subject is having preserved peripheral nerve function.

In another embodiment the present invention relates to a method of administration of a TRPA1 antagonist for relieving pain associated with diabetic peripheral neuropathy in a subject in need thereof comprising administering a therapeutically effective amount of Compound I or its pharmaceutically acceptable salt. In one aspect the therapeutically effective amount of Compound I or its pharmaceutically acceptable salt is administered in a pharmaceutical composition suitable for oral administration

In another embodiment the present invention relates to a method of administration of nanoparticulate composition comprising a therapeutically effective amount of compound I or its pharmaceutically acceptable salt for the treatment of pain associated with diabetic peripheral neuropathy in a subject in need thereof.

The pharmaceutical composition of the present invention may contain about 10 mg, or about 20 mg, or about 30 mg, or about 50 mg, or about 75 mg, or about 90 mg, or about 120 mg, or about 150 mg, or about 180 mg, or about 200 mg, or about 250 mg, or about 300 mg, or about 350 mg, or about 400 mg, or about 450 mg, or about 500 mg of Compound I or its pharmaceutically acceptable salt.

The pharmaceutical compositions for oral administration may be in various forms, for example, tablets, capsules, granules (synonymously, “beads” or “particles” or “pellets”), solution, suspension, emulsions, powders, dry syrups, and the like. In a preferred embodiment, the pharmaceutical composition for oral administration is in the form of granules or tablets or capsules.

In one embodiment, the present invention provides a pharmaceutical composition suitable for oral administration for the treatment of pain associated with diabetic peripheral neuropathy in a subject in need thereof; wherein the composition comprises from about 1 mg to about 1000 mg of Compound I or its pharmaceutically acceptable salt. In one aspect a subject is having preserved peripheral nerve function.

In one aspect the composition comprises from about 30 mg to about 800 mg or from about 60 mg to about 600 mg of Compound I or its pharmaceutically acceptable salt. In one aspect the subject is having preserved peripheral nerve function.

In one aspect of this embodiment, the pharmaceutical composition is granules or tablets or capsules. In one aspect of this embodiment, the pharmaceutical composition is a nanoparticulate composition. The nanoparticulate formulation of the present invention can be converted into a suitable pharmaceutical composition.

In another aspect the pharmaceutical composition is administered once daily or twice daily. In another aspect the pharmaceutical composition is administered once daily or twice daily for a period of at least 4 weeks, or at least 12 weeks.

Preferably, the pharmaceutical composition includes Compound I as a potassium salt.

In one embodiment, the present invention relates to a nanoparticulate pharmaceutical composition suitable for oral administration for the treatment or relief of pain associated with diabetic peripheral neuropathy in a subject in need thereof; wherein composition comprises from about 1 mg to about 1000 mg of Compound I or its salt and a surface stabilizer, said formulation having an effective average particle size in the range from about 20 nm to about 1000 nm. In aspect of this embodiment, the effective average particle size is in the range from about 30 nm to about 800 nm, or from about 50 nm to 600 nm, or from about 70 nm to about 500 nm, or from about 100 nm to 400 nm.

### DETAILED DESCRIPTION OF THE INVENTION

The present invention relates to a method of treating neuropathic pain in a subject in need thereof by administering to the subject a therapeutically effective amount of a TRPA1 antagonist.

A co-assigned PCT Application No. PCT/M2010/000930 (“the '930 application”, published as WO 2010/109334) discloses, as TRPA1 modulators, thienopyrimidinedione compounds of the formula (A):

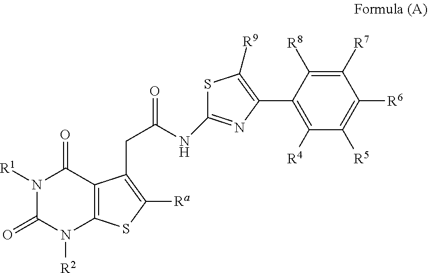

or a pharmaceutically acceptable salt thereof,
wherein

R¹, R² and R^{a}, which may be the same or different, are each independently hydrogen or (C₁-C₄)alkyl;

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

In one of the embodiment, the present invention relates to a method of treating neuropathic pain in a subject in need thereof by orally administering to the subject a therapeutically effective amount of a TRPA1 antagonist of formula (A)

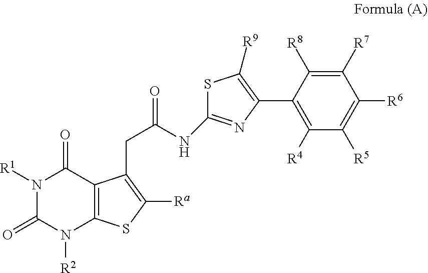

or a pharmaceutically acceptable salt thereof,
wherein

R¹, R² and R^{a}, which may be the same or different, are each independently hydrogen or (C₁-C₄)alkyl and

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

According to one embodiment, compound of the formula (A) in which R¹ and R² are methyl.

According to another embodiment, compound of the formula (A) in which R⁴, R⁵, R⁶ and R⁷ are independently selected from hydrogen, fluoro, trifluoromethyl and trifluoromethoxy.

According to yet another embodiment, compound of the formula (A) in which R⁸ is hydrogen.

According to yet another embodiment, compound of the formula (A) in which R⁹ is hydrogen.

In one embodiment the present invention relates to a method of treating neuropathic pain in a subject in need thereof by orally administering to the subject a therapeutically effective amount of a TRPA1 antagonist of formula (A)

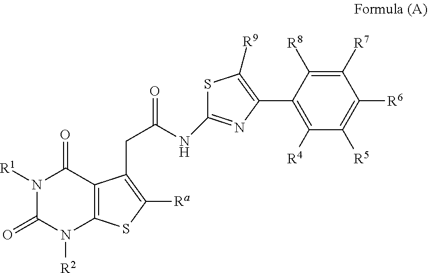

or a pharmaceutically acceptable salt thereof,
wherein

R¹ and R² are methyl;

R^{a} is hydrogen;

R⁴, R⁵, R⁶ and R⁷ are independently selected from hydrogen, fluoro, trifluoromethyl and trifluoromethoxy; and

R⁸ and R⁹ are hydrogen.

Inter alia, the '930 application discloses a compound N-{4-[2,4-difluoro-3-(trifluoromethyl)phenyl]-1,3-thiazol-2-yl}-2-(1,3-dimethyl-2,4-dioxo-1,2,3,4-tetrahydrothieno[2,3-d]pyrimidin-5-yl)acetamide (hereinafter designated as “Compound I” or its pharmaceutically acceptable salt.

### Definitions

The terms used herein are defined as follows. If a definition set forth in the present application and a definition set forth in a subsequent non-provisional application claiming priority from the present application are in conflict, the definition in the subsequent non-provisional application shall control the meaning of the terms.

The term “effective amount” or “therapeutically effective amount” denotes an amount of the TRPA1 antagonist (specifically Compound I or its pharmaceutically acceptable salt) that, when administered to a subject by oral route for treating a pain related disorder mediated by TRPA1 modulation, produces is sufficient to produce medically significant therapeutic benefit in a subject. The term “medically significant” is defined as the amount sufficient to provide at least a minimal medical benefit in the subject of administration. The effective amount of Compound I or its pharmaceutically acceptable salt to be administered per day ranges from about 1 mg to about 1000 mg, or preferably from about 30 mg to about 800 mg or more preferably from about 60 mg to about 600 mg. Typically, the therapeutically effective amount of Compound I or its pharmaceutically acceptable salt may be about 10 mg, or about 20 mg, or about 30 mg, or about 50 mg, or about 75 mg, or about 90 mg, or about 120 mg, or about 150 mg, or about 180 mg, or about 200 mg, or about 250 mg, or about 300 mg, or about 350 mg, or about 400 mg, or about 450 mg, or about 500 mg, although larger or smaller amount is not excluded if they fall within the scope of the definition of this paragraph. Subjects who do not experience sufficient benefit with a certain dose, the dose may be further increased based on efficacy and tolerability. Further, if there is no evidence that such an increase dose confers additional benefit, the dose can be reduced.

In the context of present invention, the effective amount of Compound I or its pharmaceutically acceptable salt may be administered once daily, or in divided doses two/three/four times a day. Preferably Compound I or its pharmaceutically acceptable salt may be administered once daily or twice daily.

The term “about” as used herein means an acceptable error for a particular value as determined by one of ordinary skilled in the art, which depends in part on how the value is measured or determined.

The term “active ingredient” (used interchangeably with “active” or “active substance” or “drug”) as used herein includes Compound I or its pharmaceutically acceptable salt. In one embodiment, the active ingredient includes potassium salt of Compound I.

By “an effective average particle size in the range from about 20 nm to about 1000 nm” it is meant that at least 50% of the total particles of Compound I or its salt have an average particle size in the range from about 20 nm to about 1000 nm.

The term “treating” or “treatment” as used herein also covers prophylaxis, mitigation, prevention, amelioration, suppression, alleviation of symptoms, slowing the appearance of symptoms, slowing the progression of symptoms of a disease or disorder modulated by the TRPA1 in a subject.

By the term “pain”, it is meant any condition or disease related to pain that includes but not limited to acute pain, chronic pain, mild pain, moderate pain, severe pain and can include nociceptive pain, inflammatory pain and pathological pain. Preferably, the pain includes neuropathic pain.

Neuropathic pain is a complex, chronic pain state that usually is accompanied by tissue injury. With neuropathic pain, the nerve fibers themselves may be damaged, dysfunctional, or injured. These damaged nerve fibers send incorrect signals to other pain centers. The impact of nerve fiber injury includes a change in nerve function both at the site of injury and areas around the injury. This type of pain is caused by a problem with one or more nerves themselves. The function of the nerve is affected in a way that it sends pain messages to the brain. Neuropathic pain is often described as burning, stabbing, shooting, aching, or like an electric shock. Based on its origin neuropathic pain may be divided into peripheral neuropathic pain, central neuropathic pain, or mixed (peripheral and central) neuropathic pain. Neuropathic pain includes but is not limited to polyneuropathy pain states (such as diabetic peripheral neuropathy & chemotherapy induced neuropathy), autonomic neuropathy pain states, peripheral nervous system (PNS) lesion or central nervous system (CNS) lesion or disease related pain states, polyradiculopathies of cervical, lumbar or sciatica type, cauda equina syndrome, piriformis syndrome, paraplegia, quadriplegia, pain states related to various Polyneuritis conditions underlying various infections, chemical injuries, radiation exposure, underlying disease or deficiency conditions (such as beriberi, vitamin deficiencies, hypothyroidism, porphyria, cancer, HIV, autoimmune disease like multiple sclerosis and spinal-cord injury, fibromyalgia, nerve injury, ischemia, neurodegeneration, stroke, post stroke pain, inflammatory disorders, oesophagitis, gastroeosophagal reflux disorder (GERD), irritable bowel syndrome, inflammatory bowel disease, pelvic hypersensitivity, urinary incontinence, cystitis, stomach duodenal ulcer, muscle pain, pain due to colicky, hyperalgesia (such as mechanical hyperalgesia), allodynia (such as cold allodynia).

The term “diabetic peripheral neuropathy” is synonymous to “painful diabetic peripheral neuropathy” or “painful diabetic neuropathy” or “DPN” or “diabetic peripheral neuropathic pain”.

The term “subject” includes mammals like humans and other animals, such as domestic animals (e.g., household pets including cats and dogs) and non-domestic animals (such as wildlife). Preferably, the subject is a human subject.

The “subject having preserved peripheral nerve function” is characterized to have a Type 1 or Type 2 diabetes mellitus with history of pain attributed to distal symmetric chronic sensorimotor painful polyneuropathy. The subjects have normal detection threshold to thermal perception based on quantitative sensory testing (QST). The normal cold detection threshold in foot in QST value ranges from −0.8° C. to −8.8° C. in women and −0.8° C. to −13.6° C. in men from baseline. The normal warm detection threshold in foot in QST value ranges from 1.7° C. to 11.1° C. in women and 2.3° C. to 16.7° C. in men from baseline.

By “salts” or “pharmaceutically acceptable salts”, it is meant those salts which are, within the scope of sound medical judgment, suitable for use in contact with the tissues of humans and lower animals without undue toxicity, irritation, and allergic response, commensurate with a reasonable benefit to risk ratio, and effective for their intended use. Representative acid addition salts include hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, and lauryl sulphate salts. Representative alkali or alkaline earth metal salts include the sodium, calcium, potassium and magnesium salts.

By “pharmaceutically acceptable excipient”, it is meant any of the components of a pharmaceutical composition other than the actives and which are approved by regulatory authorities or are generally regarded as safe for human or animal use.

### Methods of Treatment

In one embodiment, the present invention relates to a method of treating neuropathic pain in a subject in need thereof by orally administering to the subject a therapeutically effective amount of N-{4-[2,4-difluoro-3-(trifluoromethyl)phenyl]-1,3-thiazol-2-yl}-2-(1,3 -dimethyl-2,4-dioxo-1,2,3,4- tetrahydrothieno[2,3-d]pyrimidin-5-yl)acetamide or its pharmaceutically acceptable salt.

The therapeutically effective amount of Compound I or its pharmaceutically acceptable salt to be orally administered per day in context of the present invention is in the range from about 1 mg to about 1000 mg, or preferably from about 30 mg to about 800 mg, or more preferably from about 60 mg to about 600 mg. Typically, the therapeutically effective amount of Compound I or its pharmaceutically acceptable salt may be about 10 mg, or about 20 mg, or about 30 mg, or about 50 mg, or about 75 mg, or about 90 mg, or about 120 mg, or about 150 mg, or about 180 mg, or about 200 mg, or about 250 mg, or about 300 mg, or about 350 mg, or about 400 mg, or about 450 mg, or about 500 mg. The therapeutically effective amount of Compound I or its pharmaceutically acceptable salt may be administered to the subject in the form of a pharmaceutical composition. Compound I or its pharmaceutically acceptable salt may be orally administered for a period of at least 4 weeks, or 8 weeks, or 12 weeks, or 16 weeks or 20 weeks, or 24 weeks.

In the context of present invention, the effective amount of Compound I or its pharmaceutically acceptable salt may be orally administered as once daily, or in divided doses two/three/four times a day. Preferably Compound I or its pharmaceutically acceptable salt may be orally administered once daily or twice daily.

In one embodiment, the present invention relates to a method of treating neuropathic pain in a subject in need thereof, the said method comprising orally administering per day to the subject from about 1 mg to about 1000 mg of Compound I or its pharmaceutically acceptable salt.

In one embodiment, the present invention relates to a method of treating neuropathic pain in a subject in need thereof, the said method comprising orally administering per day to the subject from about 1 mg to about 1000 mg of Compound I or its pharmaceutically acceptable salt for a period of at least 4 weeks.

In one aspect of this embodiment the Compound I or its pharmaceutically acceptable salt is administered in an amount ranging from about 30 mg to about 800 mg, or from about 60 mg to about 600 mg daily. In another aspect of this embodiment, the Compound I or its pharmaceutically acceptable salt is administered once daily or twice daily.

In yet another aspect of this embodiment, the neuropathic pain comprises pain associated with peripheral neuropathy, diabetic peripheral neuropathy (DPN), trigeminal neuralgia, post herpetic neuralgia, visceral pain, cancer, multiple sclerosis, spinal-cord injury, fibromyalgia, stroke, inflammatory disorder, mechanical hyperalgesia, cold allodynia. Preferably the neuropathic pain comprises pain associated with peripheral neuropathy, diabetic peripheral neuropathy, post herpetic neuralgia, spinal-cord injury, fibromyalgia, mechanical hyperalgesia, and cold allodynia. Most preferably, the neuropathic pain comprises pain associated with diabetic peripheral neuropathy.

In an embodiment, the Compound I is administered in the form of its potassium salt.

In one embodiment, the present invention relates to a method of treating pain associated with diabetic peripheral neuropathy in a subject in need thereof by orally administering to the subject a therapeutically effective amount of N-{4-[2,4-difluoro-3-(trifluoromethyl)phenyl]-1,3-thiazol-2-yl}-2-(1,3-dimethyl-2,4-dioxo-1,2,3,4-tetrahydrothieno[2,3-d]pyrimidin-5-yl)acetamide or its pharmaceutically acceptable salt.

In one embodiment, the present invention relates to a method of treating pain associated with diabetic peripheral neuropathy in a subject in need thereof by orally administering to the subject a therapeutically effective amount of potassium salt of N-{4-[2,4-difluoro-3-(trifluoromethyl)phenyl]-1,3-thiazol-2-yl}-2-(1,3-dimethyl-2,4-dioxo-1,2,3,4-tetrahydrothieno[2,3-d]pyrimidin-5-yl)acetamide.

In one embodiment the present invention relates to a method of treating pain associated with diabetic peripheral neuropathy in a subject in need thereof, said method comprising orally administering per day to the subject from about 30 mg to about 800 mg, or preferably from about 60 mg to about 600 mg of Compound I or its pharmaceutically acceptable salt.

In one embodiment the present invention relates to a method of treating pain associated with diabetic peripheral neuropathy in a subject in need thereof, said method comprising orally administering per day to the subject from about 30 mg to about 800 mg, or preferably from about 60 mg to about 600 mg of Compound I or its pharmaceutically acceptable salt wherein the subject is administered once daily or twice daily.

In one embodiment the present invention relates to a method of treating pain associated with diabetic peripheral neuropathy in a subject in need thereof, said method comprising orally administering per day to the subject from about 30 mg to about 800 mg, or preferably from about 60 mg to about 600 mg of Compound I or its pharmaceutically acceptable salt for a period of at least 4 weeks.

In an aspect of this embodiment, the Compound I or its pharmaceutically acceptable salt is administered once daily or twice daily. In another aspect, the Compound I is administered in the form of its potassium salt.

In an embodiment, the present invention relates to a method of treating pain associated with diabetic peripheral neuropathy in a subject in need thereof, said method comprising orally administering to the subject Compound I or its pharmaceutically acceptable salt, wherein said method is further characterized by one or more of the followings:
A change in average pain intensity (API) ranging from about 10% to about 50%, or from about 25% to about 35% from baseline.
A change in night-time average pain intensity ranging from about 10% to about 50%, or from about 25% to about 35% from baseline.
A change in night-time worst pain intensity ranging from about 10% to about 50%, or from about 25% to about 35% from baseline.
A change in mean sleep interference score ranging from about 20% to about 60%, or from about 35% to about 45% from baseline.
A reduction in mean daily dose of rescue medication ranging from about 50% to about 90%, or from about 60% to about 80% from baseline.
An onset of sustained improvement in the 24-hour daily average pain intensity score is achieved in about 20 days or in about 18 days from the baseline.

In one aspect of this embodiment subjects showed more than 30% reduction or preferably more than 50% reduction from baseline in average pain intensity (API) score.

In one embodiment the present invention relates to a method of treating pain associated with diabetic peripheral neuropathy in a subject in need thereof, said method comprising orally administering to the subject from about 30 mg to about 800 mg, or preferably from about 60 mg to about 600 mg of Compound I or its pharmaceutically acceptable salt.

In one embodiment the present invention relates to a method of treating pain associated with diabetic peripheral neuropathy wherein the Compound I or its pharmaceutically acceptable salt is administered once daily or twice daily.

In one embodiment the present invention relates to a method of treating pain associated with diabetic peripheral neuropathy in a subject in need thereof, said method comprising orally administering to the subject from about 30 mg to about 800 mg, or preferably from about 60 mg to about 600 mg of Compound I or its pharmaceutically acceptable salt per day for a period of at least 4 weeks.

In one embodiment the present invention relates to a method of treating pain associated with diabetic peripheral neuropathy in a subject in need thereof, said method comprising orally administering to the subject Compound I or its pharmaceutically acceptable salt wherein the subject showed more than 30% reduction or preferably more than 50% reduction from baseline in average pain intensity (API) score.

In one embodiment the present invention relates to a method of treating pain associated with diabetic peripheral neuropathy in a subject in need thereof, said method comprising orally administering to the subject Compound I or its pharmaceutically acceptable salt wherein the subject has preserved peripheral nerve function.

In one aspect of this embodiment the subject to be treated for pain associated with diabetic peripheral neuropathy is characterized by one or more of the followings:
A subject having moderate to severe pain with a baseline 24-hour average daily pain intensity score of 5-8 as measured on 11-point pain intensity numeric rating scale (NRS).
A subject having preserved peripheral sensory nerves (i.e., non-denervation subgroup excluding subjects with relatively damaged peripheral sensory neurons detected by Qualitative Sensory Test (QST)).
A subject having hypersensitivity to cold stimulus and/or mechanical stimulus.

In another embodiment of the present invention, the subject to be treated for diabetic peripheral neuropathy is characterized by one or more of the followings:
A human male or female subject aged between 18-75 years.
A subject having diabetes mellitus (type 1 or 2) with a distal symmetric chronic sensorimotor painful peripheral neuropathy.
A subject having history of diabetic peripheral neuropathy for at least 6 months.
A subject having “Douleur Neuropathique en 4” (DN4) questions score of ≥4.
A subject having a baseline 24-hour average daily pain intensity score ≥4 and <9 as measured on a 11-point pain intensity numeric rating scale (NRS).
A subject having stable glycemic control (i.e., Glycosylated Hemoglobin (HbAlc) <8%) for at least one or two or three months.
A subject detected to have mechanical hyperalgesia and/or cold allodynia.

In one embodiment the present invention relates to a method of treating pain associated with diabetic peripheral neuropathy in a subject in need thereof, said method comprising orally administering to the subject about 250 mg of Compound I or its pharmaceutically acceptable salt twice daily for a period of at least 4 weeks, wherein more than 30% reduction or preferably more than 50% reduction from baseline in average pain intensity (API) score is observed in the subject.

In another aspect of this embodiment, the subject is characterized by one or more of the followings:
A subject having moderate to severe pain with a baseline 24-hour average daily pain intensity score of 5-8 as measured on a 11-point pain intensity numeric rating scale (NRS).
A subject having preserved peripheral sensory nerves (i.e., non-denervation subgroup excluding subjects with relatively damaged peripheral sensory neurons detected by Qualitative Sensory Test (QST)).
A subject having hypersensitivity to cold stimulus and/or mechanical stimulus.

In one embodiment the present invention relates to a method of treating pain associated with diabetic peripheral neuropathy in a subject in need thereof, said method comprising orally administering to the subject a therapeutically effective amount of Compound I or its pharmaceutically acceptable salt, wherein the mean steady state maximum plasma concentration (Cₘₐₓ) of the Compound I or its pharmaceutically acceptable salt in the subject is in the range of about 300 ng/ml to about 5500 ng/ml at about 0.0 hour to about 24 hours after repeat oral administration at steady state. Preferably, the mean steady state maximum plasma concentration (Cₘₐₓ) of the Compound I or its pharmaceutically acceptable salt in the subject ranges from about 1000 ng/ml to about 5300 ng/ml, or about 1800 ng/ml to about 3800 ng/ml at about 0 hour to about 24 hours after repeat oral administration, at steady state.

In another embodiment the present invention relates to a method of treating pain associated with diabetic peripheral neuropathy in a subject in need thereof, said method comprising orally administering to the subject a therapeutically effective amount of Compound I or its pharmaceutically acceptable salt, wherein the mean steady state exposure (AUC 0_24) of the Compound 1 or its pharmaceutically acceptable salt in the subject ranges from about 3,000 ng.hr/ml to about 55,000 ng.hr/ml. Preferably, the mean steady state exposure (AUC 0_24) of the Compound I or its pharmaceutically acceptable salt in the subject ranges from about 15,000 ng.hr/ml to about 45,000 ng.hr/ml, or about 25,000 ng.hr/ml to about 40,000 ng.hr/ml.

In one embodiment the present invention relates to a method of treating pain associated with diabetic peripheral neuropathy in a subject in need thereof, the said method comprising orally administering to the subject a pharmaceutical composition comprising from about 30 mg to about 800 mg, or preferably from about 60 mg to about 600 mg of Compound I or its pharmaceutically acceptable salt. In one aspect, the pharmaceutical composition comprises 250 mg of Compound I or its pharmaceutically acceptable salt.

In an aspect of this embodiment, the pharmaceutical composition is administered once daily or twice daily. In another aspect, the pharmaceutical composition comprises Compound I as potassium salt.

In an embodiment, the present invention relates to a method of treating pain associated with diabetic peripheral neuropathy in a subject having preserved peripheral nerve function, said method comprising orally administering to the subject about 250 mg of Compound I or its pharmaceutically acceptable salt twice daily for a period of at least 4 weeks followed by follow up of 2 weeks, wherein change in average pain intensity (API) ranging from about 20% to about 70%, or from about 35% to about 45% from baseline.

In one aspect of this embodiment, a subject is further characterized by one or more of the following:
A subject having baseline average pain intensity (API) score of ≥5
Cold detection threshold >18° C. at baseline as determined by QST.
Warm detection threshold <49° C. at baseline as determined by QST.

In another aspect of this embodiment subjects showed more than 30% reduction or preferably more than 50% reduction from baseline in average pain intensity (API) score.

In an embodiment, the present invention relates to a method of treating pain associated with diabetic peripheral neuropathy in a subject having preserved peripheral nerve function, said method comprising orally administering to the subject Compound I or its pharmaceutically acceptable salt once daily or twice daily for a period of at least 12 weeks.

In one embodiment the present invention relates to a method of treating pain associated with diabetic peripheral neuropathy in a subject having preserved peripheral nerve function, the said method comprising orally administering to the subject a pharmaceutical composition comprising from about 50 mg to about 1000 mg, or from about 150 mg to about 800 mg, or from about 300 mg to about 600 mg of Compound I or its pharmaceutically acceptable salt for a period of at least 12 weeks.

In an aspect of this embodiment, the pharmaceutical composition is administered once daily or twice daily. In another aspect, the pharmaceutical composition comprises Compound I as potassium salt.

In an embodiment, the present invention relates to a method of treating pain associated with diabetic peripheral neuropathy in a subject having preserved peripheral nerve function, said method comprising orally administering to the subject Compound I or its pharmaceutically acceptable salt in the form of granules or tablet formulation at three different dose levels selected from 30 mg, 90 mg, 250 mg, 500 mg and 750 mg or 1000 mg.

In an embodiment, the present invention relates to a method of treating pain associated with diabetic peripheral neuropathy in a subject having preserved peripheral nerve function, said method comprising orally administering to the subject Compound I or its pharmaceutically acceptable salt in the form of granules or tablet formulation at three different dose levels selected from 30 mg, 90 mg, 250 mg, 500 mg and 750 mg or 1000 mg once daily or twice daily.

In an embodiment, the present invention relates to a method of treating pain associated with diabetic peripheral neuropathy in a subject having preserved peripheral nerve function, said method comprising orally administering to the subject Compound I or its pharmaceutically acceptable salt in the form of granules or tablet formulation at three different dose levels selected from 30 mg, 90 mg, 250 mg, 500 mg and 750 mg or 1000 mg.

In an embodiment, the present invention relates to a method of treating pain associated with diabetic peripheral neuropathy in a subject having preserved peripheral nerve function, said method comprising orally administering to the subject Compound I or its pharmaceutically acceptable salt in the form of granules or tablet formulation at three different dose levels selected from 30 mg, 90 mg, 250 mg, 500 mg and 750 mg or 1000 mg once daily or twice daily.

In an embodiment, the present invention relates to a method of treating pain associated with diabetic peripheral neuropathy in a subject having preserved peripheral nerve function, said method comprising orally administering to the subject Compound I or its pharmaceutically acceptable salt in the form of granules or tablet formulation at three different dose levels selected from 30 mg, 90 mg, 250 mg, 500 mg and 750 mg or 1000 mg once daily or twice daily for a period of at least 12 weeks.

In an embodiment, the present invention relates to a method of treating pain associated with diabetic peripheral neuropathy in a subject having preserved peripheral nerve function, said method comprising orally administering to the subject Compound I or its pharmaceutically acceptable salt in the form of granules or tablet formulation wherein said method comprises determination of one or more of the followings:
A change in average pain intensity (API) from baseline to day 85 based on 11-point pain intensity NRS.
A change from baseline to day 15, 29, 57, 85 and 112 in
   Night-time average pain intensity
   Night-time worst pain intensity
   Mean sleep interference score
   A reduction in mean daily dose of rescue medication.

In one aspect of this embodiment subjects having preserved peripheral nerve function showed more than 30% reduction or preferably more than 50% reduction from baseline in average pain intensity (API) score.

In another embodiment of the present invention, the subject having preserved peripheral nerve function to be treated for diabetic peripheral neuropathy is characterized by one or more of the followings:
A human male or female subject aged between 18-75 years having diabetes mellitus (type 1 or 2) with a distal symmetric chronic sensorimotor painful peripheral neuropathy.
A subject having history of diabetic peripheral neuropathy for at least 6 months and no greater than 5 years.
A subject having the normal reference range 5 for thermal detection thresholds (CDT and WDT) based on QST.
A subject having “Douleur Neuropathique en 4” (DN4) questions score of ≥4.
A subject having a baseline 24-hour average daily pain intensity score ≥5 and <9 as measured on a 11-point pain intensity numeric rating scale (NRS).
A subject being treatment naïve or one with pain not adequately controlled with up to maximum of 2 medications for treatment of pain.
A subject having Glycosylated Hemoglobin (HbAlc) level up to 11% and is stable on anti-diabetic medication/s for at least 3 months prior to screening.

In one embodiment, the present invention relates to a method of treating pain associated with diabetic peripheral neuropathy in a subject having preserved peripheral nerve function, said method comprising orally administering to the subject in need thereof a therapeutically effective amount of Compound I or its pharmaceutically acceptable salt, wherein the mean steady state maximum plasma concentration (Cₘₐₓ) of the Compound I or its pharmaceutically acceptable salt in the subject is in the range of about 300 ng/ml to about 5500 ng/ml at about 0.0 hour to about 24 hours after repeat oral administration at steady state. Preferably, the mean steady state maximum plasma concentration (Cₘₐₓ) of the Compound I or its pharmaceutically acceptable salt in the subject ranges from about 1000 ng/ml to about 5300 ng/ml, or about 1800 ng/ml to about 3800 ng/ml at about 0 hour to about 24 hours after repeat oral administration, at steady state.

In another embodiment the present invention relates to a method of treating pain associated with diabetic peripheral neuropathy in a subject having preserved peripheral nerve function, said method comprising orally administering to the subject in need thereof a therapeutically effective amount of Compound I or its pharmaceutically acceptable salt, wherein the mean steady state exposure (AUC ₀_{_}₂₄) of the Compound I or its pharmaceutically acceptable salt in the subject ranges from about 3,000 ng.hr/ml to about 55,000 ng.hr/ml. Preferably, the mean steady state exposure (AUC ₀_{_}₂₄) of the Compound I or its pharmaceutically acceptable salt in the subject ranges from about 15,000 ng.hr/ml to about 45,000 ng.hr/ml, or about 25,000 ng.hr/ml to about 40,000 ng.hr/ml.

In one embodiment, the present invention relates to a method of treating diabetic peripheral neuropathy in a subject in need thereof, said method comprising orally administering to the subject a therapeutically effective amount of Compound I or its pharmaceutically acceptable salt wherein the method results in a disease modifying effect as assessed by a sustained reduction in pain for a period of at least 6 weeks after termination of the treatment.

‘Disease modifying effect’ in context of the present invention means repairing the damaged peripheral nerve fibers or preventing the progression of neuropathy or preventing the loss of small nerve fibers and thus ameliorating the root-cause at pathophysiological level rather than providing a mere symptomatic relief of the pain associated with diabetic peripheral neuropathy.

In one embodiment, the present invention relates to a method of treating pain associated with diabetic peripheral neuropathy in a subject in need thereof, said method comprising orally administering to the subject a therapeutically effective amount of Compound I or its pharmaceutically acceptable salt, wherein a subject is on stable maintenance dose of at least one another diabetic peripheral neuropathic pain medication and have moderate to severe neuropathic pain. In one aspect, the stable maintenance dose levels have been stable for at least 30 days prior to the screening visit. In one aspect, the another diabetic peripheral neuropathic pain medication may include pregabalin, gabapentin, duloxetine, Capsaicin or salts thereof.

In one embodiment, the present invention relates to a method of treating pain associated with diabetic peripheral neuropathy in a subject in need thereof, said method comprising orally administering to the subject a therapeutically effective amount of Compound I or its pharmaceutically acceptable salt, wherein a subject is nonresponsive to other diabetic peripheral neuropathic pain medication such as pregabalin, gabapentin, duloxetine, Capsaicin or salts thereof. In one aspect a subject is having preserved peripheral nerve function.

In one embodiment, the present invention relates to a method of treating pain associated with diabetic peripheral neuropathy in a subject in need thereof, said method comprising orally administering to the subject a therapeutically effective amount of Compound I or its pharmaceutically acceptable salt, wherein a subject is inadequately responsive to other diabetic peripheral neuropathic pain medication such as pregabalin, gabapentin, duloxetine, Capsaicin or salts thereof. In one aspect a subject is having preserved peripheral nerve function.

In one embodiment the present invention relates to a method of alleviating symptoms of painful diabetic peripheral neuropathy in a subject in need thereof, said method comprising orally administering to the subject therapeutically effective amount of Compound I or its pharmaceutically acceptable salt. In one aspect a subject is having preserved peripheral nerve function.

In one embodiment the present invention relates to a method of slowing the appearance of symptoms of painful diabetic peripheral neuropathy in a subject in need thereof, said method comprising orally administering to the subject therapeutically effective amount of Compound I or its pharmaceutically acceptable salt. In one aspect a subject is having preserved peripheral nerve function.

In one embodiment the present invention relates to a method of slowing the progression of symptoms of painful diabetic peripheral neuropathy in a subject in need thereof, said method comprising orally administering to the subject therapeutically effective amount of Compound I or its pharmaceutically acceptable salt. In one aspect a subject is having preserved peripheral nerve function.

In one embodiment the present invention relates to a method of treating pain associated with diabetic peripheral neuropathy in a subject in need thereof, said method comprising orally administering to the subject about 250 mg of Compound I or its pharmaceutically acceptable salt twice daily for a period of at least 4 weeks, wherein no treatment related adverse effects have been observed. In one aspect a subject is having preserved peripheral nerve function.

In an embodiment the present invention relates to a method of relieving pain associated with diabetic peripheral neuropathy by administering a therapeutically effective amount of Compound I or its pharmaceutically acceptable salt in preparation of a pharmaceutical composition suitable for oral administration in a subject in need thereof. In one aspect a subject is having preserved peripheral nerve function.

In another embodiment the present invention relates to a method of administration of a TRPA1 antagonist for relieving pain associated with diabetic peripheral neuropathy in a subject in need thereof comprising administering a therapeutically effective amount of Compound I or its pharmaceutically acceptable salt. In one aspect the therapeutically effective amount of Compound I or its pharmaceutically acceptable salt is administered in a pharmaceutical composition suitable for oral administration.

In another embodiment the present invention relates to a method of administration of nanoparticulate composition comprising a therapeutically effective amount of a TRPA1 antagonist compound I or its pharmaceutically acceptable salt for the treatment of pain associated with diabetic peripheral neuropathy in a subject in need thereof.

The treatment related adverse effects in context of the present invention may include blood and lymphatic system disorders (e.g., iron deficiency anemia, leukocytosis and neutrophilia), eye disorders (e.g., cataract and dry eye), gastrointestinal disorders (e.g., abdominal distension, diarrhoea, dyspepsia, dysphagia and gastric ulcer, haemorrhoidal haemorrhage, hyperchlorhydria, nausea and vomiting), general disorders (e.g., Fatigue, local swelling, peripheral oedema, pain and pyrexia), infections and infestations (e.g., nasopharyngitis and pharyngitis), injury, poisoning and procedural complications, contusion, change in biochemical parameters (e.g., alanine aminotransferase, aspartate aminotransferase, blood creatine phosphokinase abnormal, blood creatine phosphokinase, blood potassium, blood sodium), urine analysis, metabolism and nutrition disorders (e.g., decreased appetite, dyslipidaemia, hyperglycaemia, hypoglycaemia, hyponatraemia and impaired fasting glucose), musculoskeletal and connective tissue disorders (e.g., back pain and pain in extremity), nervous system disorders (e.g., ageusia, dysgeusia, headache and hypogeusia), psychiatric disorders (e.g., anxiety), renal and urinary disorders (e.g., diabetic nephropathy, glycosuria, pollakiuria, proteinuria and renal impairment), respiratory, thoracic and mediastinal disorders including throat irritation, skin and subcutaneous tissue disorders including skin hypopigmentation, and vascular disorders (e.g., haemorrhage, hypertension and hypotension).

The poor response to neuropathic pain therapies may reflect failure to target the individually relevant pain-generating mechanisms. Although a mechanism-based approach to treatment of neuropathic pain was suggested decades ago, very few attempts have been made to systematically determine the value of this approach. Currently there is no mechanism-based treatment available for peripheral neuropathic pain. In one embodiment, Compound I or its pharmaceutically acceptable salt is believed to relieve peripheral neuropathic pain by acting specifically by peripheral mechanism. Thus, Compound I or its pharmaceutically acceptable salt does not exhibit Central Nervous System (CNS) related adverse events like somnolence or dizziness. It is further hypothesized that Compound I or its pharmaceutically acceptable salt exhibit a disease modifying effect that prevents the progression of neuropathy.

The therapeutically effective amount of Compound I or its pharmaceutically acceptable salt may be administered per day as a single unit dose, or in multiple divided doses to the subject. The therapeutically effective amount of Compound I or its pharmaceutically acceptable salt may be administered to the subject in the form of a pharmaceutical composition.

In one embodiment, the present invention relates to a method of treating a neuropathic pain in a subject in need thereof, the said method comprising orally administering to the subject a pharmaceutical composition comprising therapeutically effective amount of Compound I or its pharmaceutically acceptable salt in an amount ranging from about 1 mg to about 1000 mg daily for a period of at least 4 weeks. In one aspect a subject is having preserved peripheral nerve function.

In an aspect of this embodiment the pharmaceutical composition comprises from about 30 mg to about 800 mg, or preferably from about 60 mg to about 600 mg of Compound I or its pharmaceutically acceptable salt to be administered per day. In another aspect of this embodiment, the pharmaceutical composition is administered once daily or twice daily. Preferably the neuropathic pain is selected from pain associated with peripheral neuropathy, diabetic peripheral neuropathy, post herpetic neuralgia, spinal-cord injury, fibromyalgia, mechanical hyperalgesia, cold allodynia. In another aspect, the pharmaceutical composition comprises Compound I as potassium salt.

In one embodiment the present invention relates to a method of treating pain associated with diabetic peripheral neuropathy in a subject having preserved peripheral nerve function, the said method comprising orally administering to the subject in need thereof a pharmaceutical composition comprising from about 30 mg to about 800 mg, or preferably from about 60 mg to about 600 mg of Compound I or its pharmaceutically acceptable salt.

In one embodiment the present invention relates to a method of treating pain associated with diabetic peripheral neuropathy in a subject having preserved peripheral nerve function, the said method comprising orally administering to the subject in need thereof a pharmaceutical composition comprising from about 30 mg to about 800 mg, or preferably from about 60 mg to about 600 mg of Compound I or its pharmaceutically acceptable salt for a period of at least 4 weeks.

In an aspect of this embodiment, the pharmaceutical composition is administered once daily or twice daily. In another aspect, the pharmaceutical composition comprises Compound I as potassium salt.

In one embodiment, the present invention relates to Compound I or its pharmaceutically acceptable salt in a therapeutically effective amount, wherein the Compound I or its pharmaceutically acceptable salt is more effective in subject having preserved peripheral nerve function compared to those subjects with progressive loss of small nerve fibers.

In an embodiment, the present invention also relates to Compound I or its pharmaceutically acceptable salt in a therapeutically effective amount for the treatment of pain associated with diabetic peripheral neuropathy in a subject in need thereof. In one aspect a subject is having preserved peripheral nerve function.

In an embodiment, the present invention relates use of a therapeutically effective amount of Compound I or its pharmaceutically acceptable salt in preparation of a pharmaceutical composition suitable for oral administration for the treatment for pain associated with diabetic peripheral neuropathy in a subject in need thereof. In one aspect a subject is having preserved peripheral nerve function.

### Pharmaceutical Compositions

The pharmaceutical compositions of the invention may be administered by oral, parenteral, inhalation, transdermal, transmucosal and nasal routes of administration among others. Preferably, the pharmaceutical composition is administered by oral route.

The pharmaceutical composition of the present invention may contain about 10 mg, or about 20 mg, or about 30 mg, or about 50 mg, or about 75 mg, or about 90 mg, or about 120 mg, or about 150 mg, or about 180 mg, or about 200 mg, or about 250 mg, or about 300 mg, or about 350 mg, or about 400 mg, or about 450 mg, or about 500 mg of Compound I or its pharmaceutically acceptable salt.

The pharmaceutical compositions for oral administration may be in various forms, for example, tablets, capsules, granules (synonymously, “beads” or “particles” or “pellets”), solution, suspension, emulsions, powders, dry syrups, and the like. In a preferred embodiment, the pharmaceutical composition for oral administration is in the form of granules or tablets or capsules.

The pharmaceutical composition may be prepared by methods known to those skilled in the art. In one embodiment, the present invention relates to a process for preparation of a pharmaceutical composition comprising Compound I or its pharmaceutically acceptable and optionally a pharmaceutically acceptable excipient, said process comprising admixing the Compound I with the pharmaceutically acceptable excipient to form a suitable pharmaceutical formulation.

The process for making the pharmaceutical composition may, for example include, (1) granulating the active ingredient with pharmaceutically acceptable carriers so as to obtain granulate, and (2) converting the formed granulate into suitable dosage forms for oral administration. These processes, as contemplated by a person skilled in the formulation art, have been incorporated herein for preparing the pharmaceutical composition of the present invention.

In one embodiment, the present invention provides a pharmaceutical composition suitable for oral administration for the treatment of pain associated with diabetic peripheral neuropathy in a subject in need thereof; wherein the composition comprises from about 1 mg to about 1000 mg of Compound I or its pharmaceutically acceptable salt. In one aspect a subject is having preserved peripheral nerve function. In one aspect the composition comprises from about 30 mg to about 800 mg or from about 60 mg to about 600 mg of Compound I or its pharmaceutically acceptable salt. In one aspect the subject is having preserved peripheral nerve function.

In one aspect of this embodiment, the pharmaceutical composition is granules or tablets or capsules. In one aspect of this embodiment, the pharmaceutical composition is a nanoparticulate composition. The nanoparticulate formulation of the present invention can be converted into a suitable pharmaceutical composition.

In another aspect the pharmaceutical composition is administered once daily or twice daily. In another aspect the pharmaceutical composition is administered once daily or twice daily for a period of at least 4 weeks, or at least 8 weeks, or at least 12 weeks, or at least 16 weeks, or at least 20 weeks, or at least 24 weeks.

Preferably, the pharmaceutical composition includes Compound I as a potassium salt.

The following examples are provided to enable one skilled in the art to practice the invention and are merely illustrative of the invention. The examples should not be read as limiting the scope of the invention.

In one embodiment, the present invention relates to a nanoparticulate pharmaceutical composition suitable for oral administration for the treatment or relief of pain associated with diabetic peripheral neuropathy in a subject in need thereof; wherein composition comprising Compound I or its salt and a surface stabilizer, said formulation having an effective average particle size in the range from about 20 nm to about 1000 nm. In aspect of this embodiment, the effective average particle size is in the range from about 30 nm to about 800 nm, or from about 50 nm to 600 nm, or from about 70 nm to about 500 nm, or from about 100 nm to 400 nm.

### EXAMPLES

### Example 1

A Phase II clinical study to evaluate efficacy, safety and tolerability of Compound I in subjects with painful diabetic neuropathy.

The study was performed in accordance with International Conference on Harmonization (ICH), Good Clinical Practice (GCP) guidelines, ethical principles as per Declaration of Helsinki and guidelines for Clinical Trials on Pharmaceutical Products in India—GCP issued by the Central Drugs Standard Control Organization, Ministry of Health, Government of India.

### Objectives

To assess the efficacy of Compound I in the treatment of pain associated with diabetic peripheral neuropathy (DPN).
To evaluate the safety and tolerability of Compound I administered once daily (OD) or twice daily (BID) in subjects with painful DPN.
To investigate effect of Compound I on time to sustained improvement in pain, night-time pain, intensity of pain on neuropathic pain symptoms inventory (NPSI), and sleep in subjects with painful DPN.
To evaluate number of subjects who are responders on the Subject Global Impression of Change (PGIC) Questionnaire, Clinician Global Impression of Change (CGIC) Questionnaire; and number of subjects who achieve various levels of percent reduction in pain.
To investigate the pharmacokinetics (PK) of Compound I in subjects with DPN
To investigate effect of Compound I on the use of rescue medication (ibuprofen 400 mg to 600 mg four times a day or paracetamol 500 mg to 1000 mg two to three times a day) in subjects with painful DPN
To investigate effect of Compound I in subjects with painful DPN who have mechanical hyperalgesia and/or cold allodynia.

### Study Design

This was a multi-center, randomized, double-blind, placebo-controlled, parallel group, 4 week study of Compound I 250 mg BID in subjects with painful DPN. The study included a total of 138 subjects with DPN, randomized in a 1:1 ratio into 250 mg BID Compound I or Placebo. The study was conducted for a total period of 9 weeks as: up to a 2-week screening/washout period, a 1-week placebo-run in period, 4 weeks of treatment period and 2 weeks of follow-up period.

Main Criteria for Inclusion of subjects were:
Men and women (post-menopausal/surgically sterile only) aged between 18-75 (both inclusive) years
Subjects having diabetes mellitus (type 1 or 2) with a distal symmetric chronic sensorimotor painful peripheral neuropathy
A history of DPN pain for at least 6 months and no greater than 5 years.
DN4 “Douleur Neuropathique en 4” questions score of ≥4
A baseline 24-hour average daily pain intensity score ≥4 and <9 as measured on a 11-point pain intensity NRS (numeric rating scale)
Subjects having stable glycemic control for at least one or two or three months prior to randomization having HbAlc (Glycosylated Hemoglobin) <8%
Subjects detected to have mechanical hyperalgesia and/or cold allodynia (detected with appropriate methodology)

Investigational product Reference product, dosage and mode of administration: Study medications were:
Placebo matching 250 mg Compound I during the placebo run-in period was taken orally BID in a fed state (30 minutes after breakfast/dinner) every day for day −7 to day −1 during Run-in period. The time interval between 2 consecutive doses must be 12 hours ±60 min
250 mg Compound I potassium granules or matching placebo during day-1 to day-28 treatment period. The time interval between 2 consecutive doses was 12 hours ±60 min.
Reference therapy: Placebo to match investigational product, administered BID.

Criteria for Evaluation: Clinical Endpoints were:
Change from baseline to end of treatment (i.e., baseline to end of week 4) in the mean 24-hour API score based on an 11-point pain intensity NRS
Change from baseline at the end of week 1, 2, 3, 4 and 6 in:
   Mean night-time API (average pain intensity) Score (subject diary): night-time is defined as the time between going to bed at night and rising in the morning
   Mean night-time worst pain intensity Score (subject diary): worst pain is defined as the subject's assessment of their worst pain intensity for the time period
   Mean sleep interference Score (subject diary): A 11-point scale that asks subjects to select the score that best describes how much the pain interfered with sleep during the past 24 hours. A score 0=Did not interfere with sleep, 10=unable to sleep due to pain
   Mean daily dose of rescue medication (subject diary)
   Number of subjects who are responders on the PGIC (Subject Global Impression of Change) Questionnaire (visits)
   Number of subjects who are responders on the CGIC (Clinician Global Impression of Change) Questionnaire (visits)
   Number of subjects achieving various levels of percent reduction from baseline in the mean API score (derived from NRS score)
   Time to onset of sustained improvement in the 24-hour daily average pain intensity Score.
   Pain intensity as assessed by the NPSI (neuropathic pain symptoms)
   QST (Quantitative sensory testing) assessments.
Change from baseline in the 24 hour daily API on NRS at the end of week 1, 3 and 6.

### Results

| | | Mean API | |
|---|---|---|---|
| Study Parameter | Time period | score | % Reduction |
| API Score | Week 0 | 6.0 | — |
| | Week 4 | 4.1 | 31.66 |
| Night-time API Score | Week 0 (baseline) | 5.1 | — |
| | Week 4 | 3.4 | 33.33 |
| Night-time Worst | Week 0 | 5.3 | — |
| pain intensity Score | (baseline) | | |
| | Week 4 | 3.5 | 33.96 |
| Sleep interference score | Week 0 (baseline) | 4.5 | — |
| | Week 4 | 2.7 | 40 |
| Change in mean daily | Week 0 (baseline) | 425 mg | — |
| dose of rescue | Week 4 | 111 mg | 73.8 |

Onset of Sustained improvement in 24-hour API Score is achieved in 18 days

Exploratory assessment of Change in 24-hour API Score from the baseline

| | | | % Reduction |
|---|---|---|---|
| Subject Characteristics/ | | Mean API | from |
| Sub-group | Time period | score | baseline |
| Moderate to severe pain | Week 0 (baseline) | 6.3 | — |
| (NRS score 5-8) | Week 4 | 4.2 | 33.33 |
| Preserved peripheral | Week 0 (baseline) | 6.2 | — |
| sensory neurons* | Week 4 | 4.1 | 33.33 |
| Moderate to severe pain | Week 0 (baseline) | 6.4 | — |
| (NRS score 5-8) and | Week 4 | 4.1 | 35.93 |
| preserved peripheral | | | |
| sensory neurons | | | |
| Hypersensitivity to cold | Week 0 (baseline) | 6.1 | — |
| stimulus and/or mechanical | Week 4 | 4.1 | 32.78 |
| stimulus | | | |
| Moderate to severe pain | Week 0 (baseline) | 6.5 | — |
| (NRS score 5-8) and hyper- | Week 4 | 4.1 | 36.92 |
| sensitivity | | | |
| to cold stimulus and/or | | | |
| mechanical stimulus | | | |

*Non-denervation subgroup excluding subjects with relatively damaged peripheral sensory neurons detected by Qualitative Sensory Test (QST)

| | % subject population at the end of | |
|---|---|---|
| | 4 week treatment | |
| | >30% Reduction | >50% Reduction |
| Subject Characteristics/ | from baseline | from baseline |
| Sub-group | API Score | API Score |
| Moderate to severe pain | 58.6 | 34.5 |
| (NRS score 5-8) | | |
| preserved peripheral | 52.9 | 35.3 |
| sensory neurons* | | |
| Moderate to severe pain | 56.7 | 40.0 |
| (NRS score 5-8) and | | |
| preserved peripheral | | |
| sensory neurons | | |
| Hypersensitivity to cold | 59.1 | 36.4 |
| stimulus and/or mechanical | | |
| stimulus | | |
| Moderate to severe pain | 66.7 | 44.4 |
| (NRS score 5-8) and | | |
| hypersensitivity to cold | | |
| stimulus and/or mechanical | | |
| stimulus | | |

*Non-denervation subgroup excluding subjects with relatively damaged peripheral sensory neurons detected by Qualitative Sensory Test (QST)

### Example 2

A Phase II clinical study to evaluate efficacy, safety and tolerability of Compound I in subjects with painful diabetic neuropathy.

### Objectives

Objectives of this study were To assess the safety, efficacy and tolerability of Compound I in the treatment of pain associated with diabetic peripheral neuropathy (DPN) and to evaluate efficacy of Compound I administered once daily (OD) or twice daily (BID) in subjects with painful DPN and having preserved peripheral nerve function and to investigate effect of Compound I on time to sustained improvement in pain, night-time pain, intensity of pain on neuropathic pain symptoms inventory (NPSI), and sleep in subjects with painful DPN and having preserved peripheral nerve function.
This was a randomized, double-blind, placebo-controlled, parallel group, 4 week treatment followed by 2 weeks follow up study of Compound I 250 mg and placebo BID in subjects with painful DPN and having preserved peripheral nerve function. The study included a total of 136 subjects with DPN and having preserved peripheral nerve function.
Clinical Endpoints were change from baseline in mean 24-hour API score based on an 11-point pain intensity NRS at the end of week 4 and week 6 in
   In ITT (Intent-to-test) population and
   In subject having one or more from baseline average pain intensity (API) score of ≥5, Cold detection threshold >18° C. at baseline as determined by QST and Warm detection threshold <49° C. at baseline as determined by QST

### Results

| | | | % Reduction |
|---|---|---|---|
| | | Mean API | from |
| Study Parameter | Time period | score | baseline |
| API Score in ITT | Week 0 (baseline) | 6.0 | — |
| population | Week 4 | 4.07 | 32.16 |
| | Week 6 | 3.59 | 40.16 |
| API Score in subject having | Week 0 (baseline) | 5.0 | — |
| one or more from baseline | Week 4 | 2.52 | 49.6 |
| average pain intensity (API) | Week 6 | 1.69 | 66.2 |
| score of ≥5, Cold detection | | | |
| threshold >18° C. at | | | |
| baseline as determined by | | | |
| QST and Warm detection | | | |
| threshold <49° C. at base- | | | |
| line as determined by QST | | | |

### Example 3

A Phase II clinical study to evaluate efficacy, safety and tolerability of Compound I in subjects with painful diabetic neuropathy in subject having preserved peripheral nerve function.

The study is performed to evaluate an effective and safe dose range of Compound I in painful DPN subjects with preserved peripheral nerve function. The study is performed at three different dose regimens of a tablet formulation for 12 weeks. The dose regimens selected are 250 mg, 500 mg and 750 mg or 1000 mg granules administered once daily or twice daily.

### Objectives

To assess efficacy of three different doses of Compound I compared with placebo in reducing pain associated with diabetic peripheral neuropathy in adult subjects with Type 1 or Type 2 diabetes mellitus with preserved peripheral nerve function.
To evaluate the following at three different doses of Compound I administered in subjects with painful DPN with preserved peripheral nerve function.
   Safety and tolerability of different doses of Compound I.
   Time to sustained improvement in pain, night-time pain, neuropathic pain symptoms inventory (NPSI), sleep interference and brief pain inventory.
   Number of subjects who are responders on the Subject Global Impression of Change (PGIC) questionnaire, Clinician Global Impression of Change (CGIC) questionnaire; and number of subjects who achieve various levels of percent reduction in pain.
   Effect of Compound I on the use of rescue medication.
   Pharmacokinetics (PK) of Compound I. To assess the efficacy of Compound I in the treatment of pain associated with diabetic peripheral neuropathy (DPN).
To explore the following at three different doses of Compound I in subjects with painful DPN with preserved peripheral nerve function.
   Effect of Compound I in subjects who have mechanical hyperalgesia and/or cold allodynia.
   Effect of Compound I in subjects who have hypersensitivity to cold pain and/or mechanical stimulus.
   Hypersensitivity to cold pain and/or mechanical stimulus is defined as one standard deviation from the mean of the reference interval.
   Efficacy of Compound I in treatment-naive subjects in comparison with subjects whose pain was uncontrolled even with up to a maximum of two approved medications at different times for the treatment of painful DPN.
   Potential disease modifying mechanism of Compound I in skin biopsy sample.
   TRPA1 polymorphism in correlation to efficacy.

### Study Population

The subject having preserved peripheral nerve function to be treated for pain associated with diabetic peripheral neuropathy is characterized by one or more of the followings:
A human male or female subject aged between 18-75 years having diabetes mellitus (type 1 or 2) with a distal symmetric chronic sensorimotor painful peripheral neuropathy.
A subject having history of diabetic peripheral neuropathy for at least 6 months and no greater than 5 years.
A subject having the normal reference range for thermal detection thresholds as Cold detection threshold (CDT) and Warm detection threshold (WDT) based on Qualitative Sensory testing (QST).
A subject having “Douleur Neuropathique en 4” (DN4) questions score of ≥4.
A subject having a baseline 24-hour average daily pain intensity score ≥5 and <9 as measured on a 11-point pain intensity numeric rating scale (NRS).
A subject being treatment naïve or one with pain not adequately controlled with up to maximum of 2 medications for treatment of pain.
A subject having Glycosylated Hemoglobin (HbAlc) level up to 11% and is stable on anti-diabetic medication/s for at least 3 months prior to screening.

### Study Design

This is a multi-center, randomized, double-blind, placebo-controlled, parallel group, 12 week study of three different doses of Compound I potassium salt in subjects with painful DPN with preserved peripheral nerve function. The study includes a total of 496 subjects with DPN with preserved peripheral nerve function randomized in a 1:1:1:1 ratio into three doses once daily or twice daily of Compound I or Placebo. The study is conducted for a total period 2 weeks of placebo run-in period, 12 weeks of double-blind treatment period and follow-up visit till 4 weeks.

### Investigational Product Reference Product, Dosage and Mode of Administration

Study medications were:
Placebo matching 250 mg, 500 mg and 750 mg or 1000 mg Compound I during the placebo run-in period was taken orally OD or BID in a fed state (30 minutes after breakfast/dinner) every day for day −14 to day −1 during Run-in period. The time interval between 2 consecutive doses must be 12 hours±60 min
Study medications are to be taken orally OD or BID in a fed state (30 minutes after breakfast/dinner) every day in the doses of 250 mg, 500 mg and 750 mg or 1000 mg Compound I potassium granules from Day 1 to Day 85. The time interval between 2 consecutive doses was 12 hours±60 min.

### Clinical Endpoints for Evaluation

A change in average pain intensity (API) from baseline to Day 85 based on 11-point pain intensity NRS.
A change from baseline to day 15, 29, 57, 85 and 112 in
   Night-time average pain intensity
   Night-time worst pain intensity
   Mean sleep interference score
   A reduction in mean daily dose of rescue medication

### Example 4

A Phase II clinical study to evaluate efficacy, safety and tolerability of Compound I in subjects with painful diabetic neuropathy in subject having preserved peripheral nerve function.

The study is performed to evaluate an effective and safe dose range of COMPOUND I in painful DPN subjects with preserved peripheral nerve function. The study is performed at three different dose regimens of a tablet formulation for 12 weeks followed by 4 weeks follow-up. The dose regimens selected are 30 mg, 90 mg and 250 mg granules administered once daily or twice daily.

### Objectives

To assess efficacy of three different doses of Compound I compared with placebo in reducing pain associated with diabetic peripheral neuropathy in adult subjects with Type 1 or Type 2 diabetes mellitus with preserved peripheral nerve function.
To evaluate the following at three different doses of Compound I administered in subjects with painful DPN with preserved peripheral nerve function.
   Time to sustained improvement in pain, night-time pain, neuropathic pain symptoms inventory (NPSI), sleep interference and brief pain inventory.
   Number of subjects who are responders on the Subject Global Impression of Change (PGIC) questionnaire, Clinician Global Impression of Change (CGIC) questionnaire; and number of subjects who achieve various levels of percent reduction in pain.
   Effect of Compound I on the use of rescue medication.
   Pharmacokinetics (PK) of Compound I. To assess the efficacy of Compound I in the treatment of pain associated with diabetic peripheral neuropathy (DPN).
To explore the following at three different doses of Compound I in subjects with painful DPN with preserved peripheral nerve function.
   Efficacy of Compound I in treatment-naive subjects and in subjects who were on DPN pain medication at screening.
   Effect of Compound I in subjects who have mechanical hyperalgesia and/or cold allodynia.
   Effect of Compound I in subjects who have hypersensitivity to cold pain and/or mechanical stimulus.
   Hypersensitivity to cold pain and/or mechanical stimulus is defined as one standard deviation from the mean of the reference interval.
   Validation of bed-side Quantitative Sensory Testing (QST) for identifying responder population in comparison with standard QST testing
   TRPA1 polymorphism in correlation to efficacy.

### Study Population

The subject having preserved peripheral nerve function to be treated for diabetic peripheral neuropathy is characterized by one or more of the followings:
A human male or female subject aged between 18-75 years having diabetes mellitus (type 1 or 2) with a distal symmetric chronic sensorimotor painful peripheral neuropathy.
A subject having history of diabetic peripheral neuropathy for at least 6 months and no greater than 5 years.
A subject having the normal reference range for thermal detection thresholds as Cold detection threshold (CDT) and Warm detection threshold (WDT) based on Qualitative Sensory testing (QST).
A subject having “Douleur Neuropathique en 4” (DN4) questions score of ≥4.
A subject having a baseline 24-hour average daily pain intensity score ≥5 and <9 as measured on a 11-point pain intensity numeric rating scale (NRS).
A subject being treatment naïve or one with pain not adequately controlled with up to maximum of 2 medications for treatment of pain.
A subject having Glycosylated Hemoglobin (HbAlc) level up to 8% and is stable on anti-diabetic medication/s for at least 3 months prior to screening.

### Study Design

This is a multi-center, randomized, double-blind, placebo-controlled, parallel group, 12 week treatment followed by 4 weeks follow-up study of three different doses of Compound I potassium salt in subjects with painful DPN with preserved peripheral nerve function. The study includes a total of 496 subjects with DPN with preserved peripheral nerve function randomized in a 1:1:1:1 ratio into three doses once daily or twice daily of Compound I or Placebo. The study is conducted for a total period 2 weeks of placebo run-in period, 12 weeks of double-blind treatment period and follow-up visit till 4 weeks.

### Investigational Product Reference Product, Dosage and Mode of Administration

Study medications were:
Placebo matching 30 mg, 90 mg and 250 mg Compound I during the placebo run-in period was taken orally OD or BID in a fed state (30 minutes after breakfast/dinner) every day for day −14 to day −1 during Run-in period. The time interval between 2 consecutive doses must be 12 hours±60 min
Study medications are to be taken orally OD or BID in a fed state (30 minutes after breakfast/dinner) every day in the doses of 30 mg, 90 mg and 250 mg Compound I potassium granules from Day 1 to Day 85. The time interval between 2 consecutive doses was 12 hours±60 min.

### Clinical Endpoints for Evaluation

A change in average pain intensity (API) from baseline to end of treatment (i.e., from Week 0 to Week 12) based on 11-point pain intensity NRS.
A change from baseline from Week 0 to Weeks 2, 4, 8, 12 and 16 in
   Night-time average pain intensity
   Night-time worst pain intensity
   Mean sleep interference score
   A reduction in mean daily dose of rescue medication
   Number of subjects achieving 30% and 50% reduction from baseline.

1-75. (canceled)

76. A method of treating neuropathic pain in a subject in need thereof comprising administering to the subject a therapeutically effective amount of a TRPA1 antagonist of formula (A): 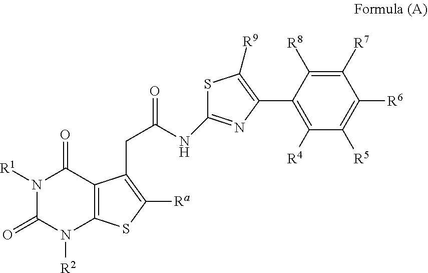
or a pharmaceutically acceptable salt thereof, wherein
R¹ and R² are methyl;
R^{a} is hydrogen;
R⁴, R⁵, R⁶ and R⁷ are independently selected from hydrogen, fluoro, trifluoromethyl or trifluoromethoxy; and
R⁸ and R⁹ is hydrogen.

77. The method of claim 76, wherein the TRPA1 antagonist is administered orally.

78. The method of claim 76, wherein from about 30 to about 800 mg of the TRPA1 antagonist is orally administered daily.

79. The method of claim 76, wherein from about 60 to about 600 mg of the TRPA1 antagonist is orally administered daily.

80. The method of claim 76, wherein the neuropathic pain comprises pain associated with peripheral neuropathy, diabetic peripheral neuropathy (DPN), trigeminal neuralgia, post herpetic neuralgia, visceral pain, cancer, multiple sclerosis, spinal-cord injury, fibromyalgia, stroke, inflammatory disorder, mechanical hyperalgesia, or cold allodynia.

81. The method of claim 76, wherein the neuropathic pain comprises pain associated with peripheral neuropathy, diabetic peripheral neuropathy, post herpetic neuralgia, spinal-cord injury, fibromyalgia, mechanical hyperalgesia, or cold allodynia.

82. A method of treating neuropathic pain in a subject in need thereof comprising administering to the subject a therapeutically effective amount of a TRPA1 antagonist, wherein the TRPA1 antagonist is N-{4-[2,4-difluoro-3-(trifluoromethyl)phenyl]-1,3-thiazol-2-yl}-2-(1,3- -dimethyl-2,4-dioxo-1,2,3,4-tetrahydrothieno[2,3-d]pyrimidin-5-yl)acetamide or a pharmaceutically acceptable salt thereof.

83. The method of claim 82, wherein the TRPA1 antagonist is a potassium salt of N-{4-[2,4-difluoro-3-(trifluoromethyl)phenyl]-1,3-thiazol-2-yl}-2-(1,3-dimethyl-2,4-dioxo-1,2,3,4-tetrahydrothieno[2,3-d]pyrimidin-5-yl)acetamide.

84. The method of claim 82, wherein the TRPA1 antagonist is administered orally.

85. The method of claim 82, wherein from about 30 to about 800 mg of the TRPA1 antagonist is orally administered daily.

86. The method of claim 82, wherein from about 60 to about 600 mg of the TRPA1 antagonist is orally administered daily.

87. The method of claim 82, wherein the neuropathic pain comprises pain associated with peripheral neuropathy, diabetic peripheral neuropathy (DPN), trigeminal neuralgia, post herpetic neuralgia, visceral pain, cancer, multiple sclerosis, spinal-cord injury, fibromyalgia, stroke, inflammatory disorder, mechanical hyperalgesia, or cold allodynia.

88. The method of claim 82, wherein the neuropathic pain comprises pain associated with peripheral neuropathy, diabetic peripheral neuropathy, post herpetic neuralgia, spinal-cord injury, fibromyalgia, mechanical hyperalgesia, or cold allodynia.
